(19) United States
(12) Patent Application Publication
(10) Pub. No.: US 2009/0011043 A1
(43) Pub. Date: Jan. 8, 2009

(54) TISSUE SEALANT MADE FROM WHOLE BLOOD

(76) Inventor: Hua Xie, Beaverton, OR (US)

Correspondence Address: KLARQUIST SPARKMAN, LLP, 121 SW SALMON STREET, SUITE 1600, PORTLAND, OR, 97204, US

(21) Appl. No.: 12/167,082

(22) Filed: Jul. 2, 2008

Related U.S. Application Data
(60) Provisional application No. 60/947,850, filed on Jul. 3, 2007.

(51) Int. Cl. A61K 35/14; A61P 7/00

(52) U.S. Cl. 424/529

(57) ABSTRACT

Disclosed are tissue sealants that include whole blood and an effective amount of an exogenous protein cross-linker to cross-link the whole blood. Also disclosed are methods for forming a tissue sealant. Such methods include, providing whole blood and mixing the whole blood with an effective amount of an exogenous protein cross-linker to cross-link the whole blood into an adherent mass, thereby forming the tissue sealant. The disclosed tissue sealants can be used for any application, for example to achieve hemostasis, bond tissue of a subject and/or or seal a fluid or gas leak in a tissue of a subject.

### CROSS REFERENCE TO RELATED APPLICATION

This application claims the benefit under 35 U.S.C. 119(e) of the earlier filing date of U.S. Provisional Application No. 60/947,850, filed Jul. 3, 2007, which is incorporated herein by reference in its entirety.

### FIELD

This application relates to tissue sealants and specifically to artificially cross-linked blood for use as a tissue sealant.

### BACKGROUND

An important aspect of medical practice is to achieve controlled hemostasis in someone who has uncontrolled bleeding caused by trauma or a medical procedure. Such hemostasis is ideally achieved by restoring tissue and vascular integrity without damaging surrounding tissue. Traditional hemostatic techniques have included applying pressure, cauterizing, suturing, and using mechanical devices such as hemostats to selectively occlude vascular flow.

Although direct application of mechanical pressure to a bleeding area with clamps, staples or sutures has been effective in staunching blood flow, these devices have a variety of limitations. They often require significant time and medical skill to apply, and are ineffectual in a number of highly vascularized organs such as the liver, lung and brain. In addition, mechanical apposition of tissue is often accompanied by leaking from the junction of the apposed tissue, and the fasteners themselves can cause trauma to surrounding tissue. To avoid such problems, efforts have been made to develop a biocompatible sealant or glue capable of bonding tissue surfaces together quickly and/or control bleeding while promoting or at least not inhibiting normal healing.

A number of tissue sealants have been developed and are currently used in various surgical disciplines. Some sealants act to stop bleeding either mechanically or by augmenting the coagulation cascade, while others are products that bind to and close defects in tissue. Existing tissue sealants for surgical use are generally divided into five major classes, including fibrin glue, bovine collagen and thrombin, cyanoacrylate, polyethylene glycol polymer, and cross-linked albumin.

Topical preparations made from synthetic biomaterials include cyanoacrylate glue (“super glue”) and polyethylene glycol polymer hydrogel. Cyanoacrylate has been used for treat urinary fistulas. Polyethylene glycol hydrogels, such as COSEAL® (Baxter, Ill.) and ADVASEAL® (Ethicon, N.J.), were approved by the FDA in 2000 as pulmonary sealants. Surgical glues have been used with success in animal partial nephrectomy models. However, most of these tissue sealants are not very effective to reverse active bleeding because the bleeding prevents contact of the agent to the tissue surface.

The concept of using clotting substances from human blood for wound management and to achieve hemostasis in bleeding from parenchymatous organs was introduced in early 20th century, when Bergel reported the hemostatic effect of fibrin. Fibrin glue mimics the final steps of the coagulation cascade. In the presence of thrombin, fibrinogen is converted to fibrin to achieve hemostasis. The thrombin and fibrinogen may be delivered from a double-barrel syringe onto a dry tissue bed where they interact to form the fibrin and provide a biological seal to the blood vessels to stop the flow of blood.

Commonly used protein based sealants that act as hemostatic agents include gelatin matrix-thrombin sealants, such as FLOSEAL® (Baxter, Ill.), and cross-linked albumin agents, such as BIOGLUE® (Cryolife, Ga.). FLOSEAL® is composed of a specially engineered bovine-derived gelatin granular matrix and thrombin. Both components work independently and synergistically to promote clot formation at the targeted bleeding site. Upon contact with blood the granules swell approximately 10%-20% to produce a tamponade effect. Clotting is enhanced with exposure to thrombin and the granule-gelatin matrix provides a framework for clot development. BIOGLUE® is a 2-component system that consists of purified bovine serum albumin and glutaraldehyde.

U.S. Pat. No. 5,385,606 discloses a bio-adhesive composition that contains serum albumins or solid mixtures obtained by dehydrating blood plasma or serum. That patent defines the “plasma” as whole blood without the cellular components, and “serum” as plasma that has been treated to prevent agglutination by removal of fibrinogen and/or fibrin.

Since the components of many currently available tissue sealants are derived from animal and human proteins, the theoretical risk of viral disease transmission from these formulations and documented anaphylactic reactions have led to concern among clinicians.

### SUMMARY

The present disclosure relates to tissue sealants, methods of making tissue sealants, and the use thereof. The new tissue sealants take advantage of the surprising finding that whole blood (including cross-linked whole blood) provides unexpectedly superior tissue sealing (including hemostasis and adhesion of tissue) as compared to prior sealants, such as serum sealants. The disclosed tissue sealants include whole blood, such as concentrated whole blood and/or autologous whole blood obtained from a subject, and an exogenous protein cross-linker, wherein the exogenous protein cross-linker is present in an amount effective to cross-link the whole blood to form an adherent (for example hemostatic) mass.

As disclosed herein a variety of exogenous protein cross-linkers can be used in the disclosed tissue sealants to effectively cross-link the protein components in whole blood. In some examples, the disclosed sealants contain an exogenous protein cross-linker that contains one or more functional groups capable of forming a covalent bond with an amine, one or more groups capable of forming a covalent bond with a sulfhydryl group, one or more groups capable of forming a covalent bond with a carboxylic acid group, or any combination thereof. In particular embodiments, the disclosed sealants contain an aldehyde as an exogenous protein cross-linker, such as a di- or polyaldehyde, for example glutaraldehyde, such as a solution of about 5% glutaraldehyde to about 10% glutaraldehyde.

Methods are also disclosed for forming tissue sealants. The disclosed methods for preparing the sealant include providing whole blood, such as concentrated and/or autologous whole blood, and mixing the whole blood with an amount of an exogenous protein cross-linker effective to cross-link the whole blood into an adherent (for example hemostatic) mass, thereby forming the tissue sealant. The tissue sealant may be formed on a target tissue of a subject, for example to act as a surgical sealant during an invasive procedure. Typically, the whole blood and the exogenous protein cross-linker are mixed and applied to the tissue of a subject where it forms an adherent (for example hemostatic) mass by forming a number of cross-links between the proteins present in the whole blood and the exogenous protein cross-linker.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic representation of an exemplary clot formed from cross-linked whole blood.

FIG. 2 is a schematic representation of a tissue sealant formed from cross-linked whole blood bonding portions of tissue.

FIG. 3 is a schematic representation of a clot formed from glutaraldehyde cross-linked whole blood.

FIG. 4 is a schematic representation of an exemplary device for concentrating whole blood.

FIG. 5 is a schematic representation of an exemplary device for delivery of whole blood and cross-linking agent to a target site, such as a wound.

FIG. 6 is a bar graph depiction the sealant strength of different cross-linked and uncross-linked blood species.

### DETAILED DESCRIPTION

### I. Terms and Examples

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “an exogenous protein cross-linker” includes single or plural exogenous protein cross-linkers and can be considered equivalent to the phrase “at least one exogenous protein cross-linker.”

As used herein, the term “comprises” means “includes.” Thus, “comprising whole blood” means “including whole blood” without excluding other elements.

Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the invention, the following explanations of terms are provided:

Acetic acid or ethanoic acid: An organic carboxylic acid with the formula CH₃COOH. Acetic acid can be used in a hypotonic solution, for example a hypotonic solution useful for lysing cells present in whole blood.

Aldehyde: An organic compound containing at least one terminal carbonyl group, also called an aldehyde, formyl, or methanoyl group. A dialdehyde is an organic compound containing two aldehyde groups. Aldehydes are amine reactive and can be used to cross-link proteins and/or cells containing an amine, for example proteins and/or cells present in whole blood. Examples of dialdehydes include glyoxal, glutaraldehyde, adipaldehyde, succinaldehyde, and suberaldehyde. In one example, an aldehyde is glutaraldehyde. Glutaraldehyde is a dialdehyde that can be used as an amine-reactive homobifunctional protein cross-linker. Monomeric glutaraldehyde can polymerize by an aldol condensation reaction yielding alpha,beta-unsaturated poly-glutaraldehyde.

Artificial clot: A blood clot formed by artificial means, for example an artificial clot can be formed from cross-linked whole blood, such as by cross-linking the proteins and/or cells present in whole blood, for example by cross-linking the proteins present in the whole blood with an exogenous protein cross-linker.

Autologous: Cells, tissues (such as blood, for example whole blood), or proteins that are reimplanted in the same subject as they come from. In one example, autologous whole blood is whole blood obtained from a subject and used (for example in the formation of a hemostatic mass) in the same subject.

Blood coagulation: The process by which blood forms solid clots, for example as part of hemostasis whereby a damaged blood vessel wall is covered by a platelet- and fibrin-containing clot to stop bleeding and begin repair of the damaged vessel. In mammals, blood coagulation typically involves both cells and proteins, such as coagulation factors. Blood coagulation is initiated almost instantly after an injury to the blood vessel damages the endothelium (lining of the vessel). Platelets form a hemostatic plug at the site of injury; this is called primary hemostasis. Secondary hemostasis occurs substantially simultaneously and involves proteins in the blood plasma, called coagulation factors, responding in a complex cascade to form fibrin strands that strengthen the platelet plug.

Coagulation factors: Proteins involved in a sequential reaction or coagulation cascade. Coagulation factors include factor I (also known as fibrinogen), factor II (also known as prothrombin), factor III, calcium, factor V (also known as proaccelerin), factor VI, factor VII, factor VIII, factor IX (also known as Christmas factor), factor X (also known as Stuart-Prower factor), factor XI, factor XII (also known as Hageman factor), factor XIII (also known as fibrin-stabilizing factor), von Willebrand factor, prekallikrein, high molecular weight kininogen, fibronectin, and plasminogen amongst others.

Coagulation cascade: A step by step process that occurs when a blood vessel is injured. The end result of the coagulation cascade is a blood clot that creates a barrier over the injury site, protecting it until it heals.

The coagulation cascade of secondary hemostasis has two pathways, the Contact Activation pathway (also known as the intrinsic pathway) and the Tissue Factor pathway (also known as the extrinsic pathway) that lead to fibrin formation. The pathways are a series of reactions, in which a zymogen (inactive enzyme precursor), typically a serine protease and its glycoprotein co-factor, are activated to become active components that then catalyze the next reaction in the cascade, ultimately resulting in cross-linked fibrin. Coagulation factors are generally indicated by Roman numerals, with a lowercase “a” appended to indicate an active form.

Concentrate: To remove non-active liquid parts (such as water) or low molecular weight parts from a liquid to provide a concentrated liquid (such as blood).

Effective amount: An amount of agent required to achieve a desired result. For example, the amount of a protein cross-linker needed to cross-link whole blood to form a hemostatic mass.

Electromagnetic radiation: A series of electromagnetic waves that are propagated by simultaneous periodic variations of electric and magnetic field intensity, and that includes radio waves, infrared, visible light, ultraviolet light, X-rays and gamma rays. In particular examples, electromagnetic radiation is emitted by a laser, which can possess properties of monochromaticity, directionality, coherence, polarization, and intensity. In some examples, electromagnetic radiation can be used as a heat source.

Exogenous: An “exogenous” agent is one that originates outside the subject. In one example, an exogenous clotting factor is a clotting factor that does not originate from the whole blood to which it is added. In another example, an exogenous protein cross-linker is a protein cross-linker that does not originate from the blood to which it is added.

Hemostatic mass: A mass of chemically cross-linked whole blood proteins and/or cells that can be used as a hemostatic seal to stop a fluid and/or gas leak in a tissue, such as a subject's tissue (including from blood vessels).

Hemostasis: The cessation or reduction of blood loss from a damaged blood vessel.

Hemostatic composition: A composition capable of promoting hemostasis.

Hemostatic seal: A seal that inhibits the loss of blood from the region of the seal. In one example, a hemostatic seal is formed from a hemostatic mass composed of cross-linked whole blood, such as cross-linked proteins and/or cells from whole blood.

Hypotonic solution: A solution containing a low concentration of solute relative to another solution (for example a low concentration of solute relative to the solute present in a cell's cytoplasm, such as the cytoplasm of a blood cell). When a cell is placed in a hypotonic solution, the water diffuses into the cell, which can cause the cell to swell and explode. In one example, a hypotonic solution is a solution from about 0.1% acetic acid to about 4% acetic acid.

Ionized argon beam: A stream of ionized argon gas that can deliver electromagnetic energy to a surface, such as a tissue surface. The flowing argon gas also can serve to clear fluids from a tissue and/or cool a tissue surface. In one example, an ionized argon beam is emitted from an argon beam coagulator.

Lysing: The process of rupturing a cell, for example by osmotic and/or mechanical mechanisms that compromises the integrity of the cellular membrane. Cytolysis is the lysis of cells in a hypotonic environment.

Peptide/Protein/Polypeptide: All of these terms refer to a polymer of amino acids and/or amino acid analogs that are joined by peptide bonds or peptide bond mimetics. The twenty naturally occurring amino acids and their single-letter and three-letter designations known in the art.

Protein cross-linker: A homo- or hetero-multifunctional reagent with at least two identical or non-identical groups that are reactive to functional group present in proteins, such as sulfhydryls and/or amine groups. In some examples, a protein cross-linker is amine reactive, meaning it is capable of forming a covalent bond with an amine group, such as an amine group present in a protein, for example amine group present on a lysine residue. Examples of amine reactive groups include aryl azides, carbodiimides, phosphines, imidoesters, N-hydroxysuccinimide-esters (NHS-esters) pentafluorophenyl-esters (PFP-esters), and vinyl sulfones amongst others. In some examples, a protein cross-linker is sulfhydryl reactive, meaning it is capable of forming a covalent bond with sulfhydryl, such as a sulfhydryl group present in protein, for example a sulfhydryl group present on a cysteine residue. Examples of sulfhydryl reactive groups include maleimides, pyridyl disulfides, and vinyl sulfones amongst others. In some examples, a protein cross-linker is carboxylic acid reactive, meaning it is capable of forming a covalent bond with a carboxylic acid group, such as carboxylic acid group present in a protein, for example a carboxylic acid group present in an aspartic acid or glutamic acid residue. Examples of carboxylic acid reactive groups include carbodiimides amongst others.

Examples of protein cross-linkers that can be used in the disclosed methods and compositions include without limitation bis(sulfosuccinimidyl) suberate (BS3), bis[2-(succinimidooxycarbonyloxy)ethyl]sulfone, dimethyl adipimidate (DMA), dimethyl pimelimidate (DMP), dimethyl suberimidate (DMS), disuccinimidyl glutarate (DSG), dithiobis(succinimidyl) propionate (DSP), disuccinimidyl tartrate (DST), dimethyl 3,3′-dithiobispropionimidate (DTBP), 3,3′-dithiobis(sulfosuccinimidylpropionate) (DTSSP), tris(succinimidyl)aminotriacetate (TSAT), EGS, Sulfo-EGS, molecules with hydroxymethyl phosphine functional groups such as THP, sulfhydryl reactive groups, such as maleimides, for example 1,4-bis(maleimido)butane (BMB), 1,4 bis-maleimidyl-2,3-dihydroxybutane (BMDB), bismaleimidohexane (BMH), bis-maleimidoethane (BMOE), dithio-bismaleimidoethane (DTME) sulfosuccinimidyl 4-N-maleimidomethyl cyclohexane-1-carboxylate (Sulfo-SMCC), and sulfosuccinimidyl 4-N-maleimidomethyl cyclohexane-1-carboxylate (SMCC), 1,4-Di-[3′-(2′-pyridyldithio)-propionamido]butane (DPDPB), sulfones such as 1,6-hexane-bis-vinylsulfone (HBVS), (tris[2-maleimido ethyl]amine) (TMEA), (3-[(2-amino ethyl)dithio]propionic acid) (AEDP), 4-[p-azidosalicylamido]butylamine, succinimidyl 6-(3-[2-pyridyldithio]-propionamido)hexanoate (LC-SPDP), LC-SMCC, SPDP, Sulfo-EMCS, Sulfo-GMBS, GMBS, Sulfo-KMUS, Sulfo-LC-SMPT, SMPT, Sulfo-MBS, MBS, Sulfo-SIAB, SIAB, Sulfo-SMPB, SMPB, AMAS, APDP, BMPS, EMCA, KMUA, SBAP, SIA, SMPH, carboiimides such as 1-ethyl-3-(3-dimethylaminopropyl)carbodiimide hydrochloride, and 1-cyclohexyl-3-(2-morpholinyl-4-ethyl)carbodiimide sulfonate, 1,3-di-p-tolylcarbodiimide; 1,3-diisopropylcarbodiimide, 1,3-dicyclohexylcarbodiimide, 1-cyclohexyl-3-(2-morpholinoethyl) carbodiimide metho-p-toluenesulfonate, polycarbodiimide, 1-tert-butyl-3-ethylcarbodiimide, 1,3-dicyclohexylcarbodiimide; 1,3-bis(trimethylsilyl)carbodiimide, 1,3-di-tert-butylcarbodiimide, 1-(3-dimethylaminopropyl)-3-ethylcarbodiimide methiodide, 1-(3-dimethylaminopropyl)-3-ethylcarbodiimide, and 1-[3-(dimethylamino)propyl]-3-ethylcarbodiimide methiodide, and aldehydes such as glyoxal, glutaraldehyde, adipaldehyde, succinaldehyde, and suberaldehyde. Additional protein cross-linkers are commercially available from Pierce Biotechnology, (Rockford, Ill.), Molecular Probes (Eugene, Oreg.), and Sigma-Aldrich (St. Louis, Mo.).

Sealing: The process of closing a leak in a tissue, such as a fluid or gas leak in a tissue, or adhering tissues to one another. A sealant need not provide a complete closure or render a target impermeable; partially closing the surface or providing a partial closure is sufficient. Examples of a sealant are compositions that inhibit a leak of liquid (such as blood) from blood vessels, fluid (such as air) from a closed viscus or body cavity, or adhere body parts (such as apposed segments of skin or organ) to one another.

Subject: Living multi-cellular vertebrate organisms, a category that includes human and non-human mammals.

Surgical incision: The cutting of or into body tissues or organs, and separating partial tissue from the tissue or organs, for example by a surgeon as part of an operation. In some examples, a surgical incision results in bleeding from the site of the surgical incision.

Tissue: A collection of interconnected cells that perform a similar function within an organism.

Tissue sealant: A sealant used to provide a seal in tissue, where the seal need not be perfect or render the target tissue completely impermeable. A surgical sealant is a tissue sealant used in a medical procedure, such as a surgical procedure to close or adhere tissue.

Whole blood: Blood containing its cellular constituents (red blood cells, white blood cells, and platelets), for example blood from which the cellular constituents have not been substantially removed. An example of whole blood is blood drawn directly from the body that has not been separated into separate components. Whole blood contains several types of cells suspended in a fluid medium known as plasma. The cellular constituents of whole blood include red blood cells (erythrocytes), white blood cells (leukocytes), and platelets (thrombocytes). In some embodiments, whole blood is concentrated whole blood in which some fraction of water has present in the unconcentrated whole blood has been removed. In some embodiments, at least some of the cells present in whole blood are lysed, for example lysed by exposure to a hypotonic solution.

Wound: A type of physical trauma wherein the skin is torn, cut or punctured (an open wound), or where blunt force trauma causes a contusion (a closed wound). In some examples, a wound is a surgical incision.

### II. Overview of Several Embodiments

Uncontrolled blood flow is a serious medical problem that can lead to morbidity and even death. For example, hemorrhage from visceral parenchymal organs such as liver, spleen and kidney is frequently difficult to control, especially in patients with complicated traumatic injuries. Visceral organ injury is also the second most common complication during laparoscopic surgery following vascular injury. Hemorrhage from the liver and other solid encapsulated organs, such as the spleen and kidney, present unique challenges to the surgeon because the lack of internal structural elements causes sutures to hold poorly.

Hemostatic agents and tissue sealants are used extensively across a broad range of surgical procedures, especially in minimally invasive and technically challenging operations. Hemostatic agents and tissue sealants typically come from five families, and include fibrin glue, cyanoacrylate, bovine collagen and thrombin, polyethylene glycol polymer, and albumin cross-linked with glutaraldehyde. One concern about these agents is that many of these protein components are derived from animal sources that may cause severe allergic reactions or transmission of blood-borne diseases to the subject who receives the treatment.

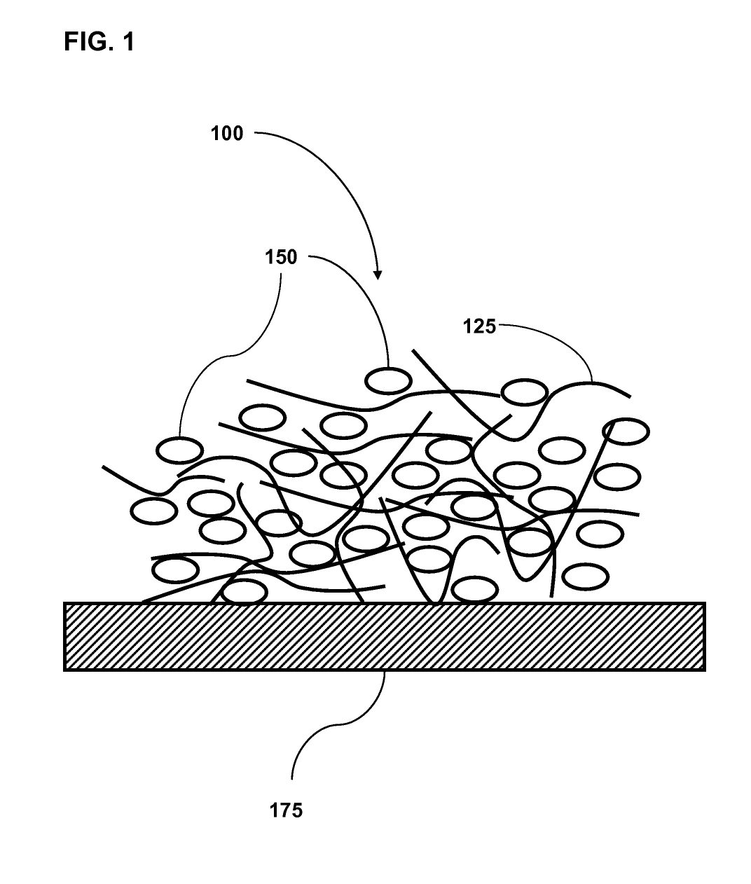

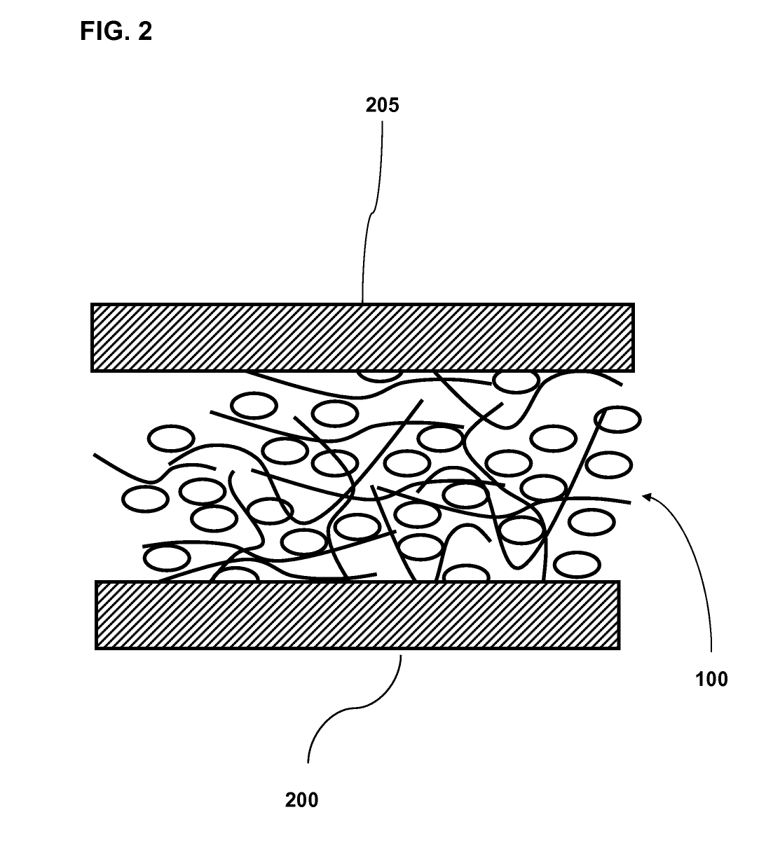

Disclosed herein is the discovery that whole blood, such as concentrated whole blood obtained from a subject, can be artificially cross-linked at the protein level using exogenous protein cross-linkers to form a durable tissue sealant that can be used to effectively control bleeding from a subject's tissue, such as bleeding from the liver or kidney of a subject. With reference to FIG. 1, an adherent mass 100 is formed from cross-linked proteins 125 and/or cells 150 on a tissue 175. The formation of mass 100 is effective in inhibiting hemorrhaging from tissue 175. With reference to FIG. 2, mass 100, can also be used to join two or more portions of tissue 200, 205, for example when used to close a wound, such as a surgical incision. While the particular examples herein disclose controlling bleeding from visceral parenchymal organs, the disclosed methods and compositions are applicable to any medical procedure wherein hemostasis or tissue bonding is desirable. These medical procedures include, without limitation, stopping uncontrolled bleeding caused by trauma (such as injury induced by an automobile accident or battlefield injury). The “medical procedures” may be performed by anyone capable of carrying them out, such as physicians (including veterinary physicians), nurses, physician assistants, paramedics, or even self-administration by an injured or bleeding person.

The disclosed tissue sealants provide certain performance advantages over existing surgical hemostatic products, such as, safe and effective hemorrhage control, quick and easy onsite preparation, simple and convenient handling, and potential low cost. In addition, the disclosed tissue sealants can be prepared quickly using autologous blood obtained from a subject undergoing a medical procedure. Thus, the disclosed tissue sealants lessen the chances of contracting a disease or initiating an immune response from the use of non-autologous agents, such as proteins derived from animal origins.

### Tissue Sealants

Disclosed herein are tissue sealants, such as hemostatic agents, that include as constituents whole blood, such as concentrated and/or autologous whole blood, and an effective amount of an exogenous protein cross-linker to cross-link proteins present in the whole blood, for example proteins such as hemoglobins, albumins, immunoglobins, cell surface proteins, growth factors, peptides and the like present in whole blood.

In some embodiments, the disclosed tissue sealant includes a volume of whole blood, such as concentrated and/or autologous whole blood, that is from about 1 times or less to about 10 times greater than the volume of the exogenous protein cross-linker, such as about 1.0 times greater, about 1.1 times greater, about 1.2 times greater, about 1.3 times greater, about 1.4 times greater, about 1.5 times greater, about 31.6 times greater, about 1.7 times greater, about 1.8 times greater, about 1.9 times greater, about 2.0 times greater, about 2.1 times greater, about 2.2 times greater, about 2.3 times greater, about 2.4 times greater, about 2.5 times greater, about 2.6 times greater, about 2.7 times greater, about 2.8 times greater, about 2.9 times greater, about 3.0 times greater, about 3.1 times greater, about 3.2 times greater, about 3.3 times greater, about 3.4 times greater, about 3.5 times greater, about 3.6 times greater, about 3.7 times greater, about 3.8 times greater, about 3.9 times greater, about 4.0 times greater, about 4.1 times greater, about 4.2 times greater, about 4.3 times greater, about 4.4 times greater, about 4.5 times greater, about 4.6 times greater, about 4.7 times greater, about 4.8 times greater, about 4.9 times greater, about 5.0 times greater, about 5.1 times greater, about 5.2 times greater, about 5.3 times greater, about 5.4 times greater, about 5.5 times greater, about 5.6 times greater, about 5.7 times greater, about 5.8 times greater, about 5.9 times greater, about 6.0 times greater, about 6.1 times greater, about 6.2 times greater, about 6.3 times greater, about 6.4 times greater, about 6.5 times greater, about 6.6 times greater, about 6.7 times greater, about 6.8 times greater, about 6.9 times greater, about 7.0 times greater, about 7.1 times greater, about 7.2 times greater, about 7.3 times greater, about 7.4 times greater, about 7.5 times greater, about 7.6 times greater, about 7.7 times greater, about 7.8 times greater, about 7.9 times greater, 8.0 times greater, about 8.1 times greater, about 8.2 times greater, about 8.3 times greater, about 8.4 times greater, about 8.5 times greater, about 8.6 times greater, about 8.7 times greater, about 8.8 times greater, about 8.9 times greater, about 9.0 times greater, about 9.1 times greater, about 9.2 times greater, about 9.3 times greater, about 9.4 times greater, about 9.5 times greater, about 9.6 times greater, about 9.7 times greater, about 9.8 times greater, about 9.9 times greater or about 10.0 times greater. In other words, the disclosed tissue sealants contain whole blood, such as concentrated and/or autologous whole blood, mixed with exogenous protein cross-linker in a volume of whole blood to exogenous cross-linker ratio from about 1 to 1 to about 10 to 1.

The disclosed tissue sealants contain an effective amount of an exogenous protein cross-linker to effectively cross-link the protein components present in whole blood, such as whole blood obtained from a subject, for example autologous whole blood obtained from a subject. A variety of exogenous protein cross-linkers can be used in the disclosed agents to effectively cross-link the protein components in whole blood. Typically, exogenous protein cross-linkers are multi-functional, such as bi-functional, tri-functional, etc., in that they have more than one functional group capable of forming a covalent bond with functional groups present in proteins, although uni-functional protein cross-linkers such as formaldehyde can also be used. In some examples, an exogenous protein cross-linker is amine reactive, meaning it is capable of forming a covalent bond with an amine group, such as an amine group present on a lysine residue, for example a lysine residue present in a protein, such as a protein present in whole blood. Examples of amine reactive groups include aldehydes, aryl azides, carbodiimides, phosphines, imidoesters, N-hydroxysuccinimide-esters (NHS-esters) pentafluorophenyl-esters (PFP-esters), and vinyl sulfones amongst others. In some examples, an exogenous protein cross-linker is sulfhydryl reactive, meaning it is capable of forming a covalent bond with a sulfhydryl group, such as a sulfhydryl group present on a cysteine residue, for example a cysteine residue present in a protein, such as a protein present in whole blood. Examples of sulfhydryl reactive groups include maleimides, pyridyl disulfides, and vinyl sulfones amongst others. In some examples, an exogenous protein cross-linker is carboxylic acid reactive, meaning it is capable of forming a covalent bond with a carboxylic acid group, such as a carboxylic acid group present in an aspartic acid or glutamic acid residue, for example an aspartic acid or glutamic acid residue contained within a protein, such as a protein present in whole blood. Examples of carboxylic acid reactive groups include carbodiimides amongst others.

In some examples, the exogenous protein cross-linker present in the composition is hetero-functional, meaning that the exogenous protein cross-linker contains more than one type of functional group, for example an exogenous protein cross-linker can contain one or more functional groups capable of forming a covalent bond with an amine, one or more groups capable of forming a covalent bond with a sulfhydryl group, one or more groups capable of forming a covalent bond with a carboxylic acid group, or any combination thereof. In some embodiments, the exogenous protein cross-linker is homo-functional, meaning that the exogenous protein cross-linker contains only one type of functional group, for example a exogenous protein cross-linker can contain one or more functional groups capable of forming a covalent bond with an amine, one or more groups capable of forming a covalent bond with a sulfhydryl group, or one or more groups capable of forming a covalent bond with a carboxylic acid group.

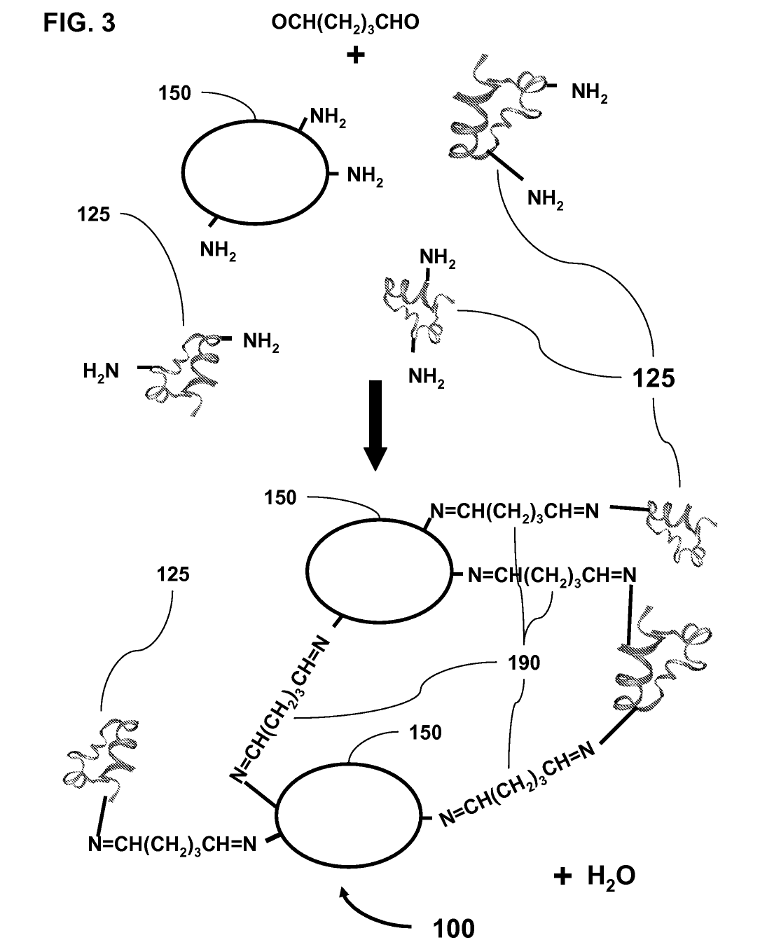

In some embodiments, the exogenous protein cross-linker contains an aldehyde, such as a di- or polyaldehyde. In some embodiments, the exogenous protein cross-linker includes glutaraldehyde. In some examples, the exogenous protein cross-linker includes a solution of about 1% or less glutaraldehyde to about 10% or more glutaraldehyde, such as a solution of about 1%, about 2%, about 3%, about 4%, about 6%, about 7%, about 8%, about 9%, or even about 10% glutaraldehyde. In some examples, the glutaraldehyde solution is a solution containing glutaraldehyde in water. With reference to FIG. 3 and the exemplary protein cross-linker glutaraldehyde, when glutaraldehyde (chemical formula OCH(CH₂)₃CHO) is mixed with proteins 125 and cells 150 containing free amine groups (NH₃) cross-links 190 are formed, thereby forming mass 100.

In some embodiments, the disclosed composition includes concentrated whole blood, such as autologous whole blood. Concentrated whole blood has a reduced volume relative to whole blood obtained from a subject, while retaining about the same amount of proteinatious material as whole blood prior to concentration. In other words, concentrated whole blood typically has at least some fraction of water removed. In addition to water, other low molecular weigh components of whole blood can be removed from concentration whole blood, for example dissolved salts or sugars and the like. In some embodiments, the concentrated whole blood has a volume that is reduced from about 1.1 times to about 10 times or more relative to the volume of the blood obtained from a subject, such as a volume that is about 1.1 times, about 1.5 times, about 2.0 times, about 2.5 times, about 3.0 times, about 3.5 times, about 4.0 times, about 4.5 times, about 5.0 times, about 5.5 times, about 6.0 times, about 6.5 times, about 7.0 times, about 7.5 times, about 8.0 times, about 8.5 times, about 9.0 times, about 9.5 times, or even 10 times reduced relative to the volume of the blood obtained from the subject.

The disclosed compositions typically have total protein concentration in whole blood of greater than 25 grams/deciliter, for example between about 25 grams/deciliter to about 100 grams/deciliter. Accordingly, the whole blood, such as concentrated and/or autologous whole blood, used in the disclosed examples of the tissue sealants has a protein concentration that is from about 25 grams/deciliter to about 100 grams/deciliter, such as about 25 grams/deciliter, about 30 grams/deciliter, about 35 grams/deciliter, about 40 grams/deciliter, about 45 grams/deciliter, 50 grams/deciliter, about 55 grams/deciliter, about 60 grams/deciliter, about 65 grams/deciliter, about 70 grams/deciliter, about 75 grams/deciliter, about 80 grams/deciliter, about 85 grams/deciliter, about 90 grams/deciliter, about 95 grams/deciliter, or about 100 grams/deciliter, for example about 25 grams/deciliter to about 100 grams/deciliter, about 25 grams/deciliter to about 75 grams/deciliter, about 25 grams/deciliter to about 50 grams/deciliter, about 50 grams/deciliter to about 100 grams/deciliter, or about 75 grams/deciliter to about 100 grams/deciliter.

In some embodiments, the disclosed compositions include whole blood in which at least some of the cells are lysed. Lysed cells have effectively spilled their protein contents, thus making these protein contents available react with the exogenous protein cross-linker present in the tissue sealant and participate in the formation of a hemostatic mass.

The disclosed compositions can optionally include other agents, such as pH modifiers, surfactants, antioxidants, osmotic agents, preservatives, drugs, and other active agents, such as exogenous agents, for example exogenous clotting factors. Examples of pH modifiers that can be included in the disclosed tissue sealants include acetic acid, boric acid, hydrochloric acid, sodium acetate, sodium bisulfate, sodium borate, sodium bicarbonate, sodium citrate, sodium hydroxide, sodium nitrate, sodium phosphate, sodium sulfite, and sulfuric acid amongst others. Examples of surfactants that can be included in the disclosed tissue sealants include, but are not limited to, ionic surfactants, such as anionic surfactants, for example sodium stearate, sodium dodecyl sulfate, α-olefinsulfonate, and sulfoalkylamides; and cationic surfactants, for example alkyldimethylbenzylammonium salts, alkyltrimethylammonium salts and alkylpyridinium salts; amphoteric surfactants such as imidazoline surfactants; and/or non-ionic surfactants including, for example, polyethylene oxide alkyl ethers, polyethylene oxide alkylphenyl ethers, glycerol fatty acid esters, sorbitan fatty acid esters, sucrose fatty acid esters, and the like. Examples of electrolytes that can be included in the disclosed tissue sealants include without limitation physiological salts, such as sodium chloride, potassium chloride and the like. Examples of preservatives that can be included in the disclosed tissue sealants include without limitation chlorobutanol, sorbate, benzalkonium chloride, parabens, chlorhexadines, and the like.

In some applications, it can be advantageous to include exogenous coagulation factors in the disclosed composition, for example to enhance the formation of a hemostatic mass. Thus, in certain embodiments, one or more exogenous coagulation factors are present in the tissue sealant, for example to initiate the natural coagulation cascade. In some embodiments, the one or more exogenous coagulation factors includes at least one of factor I (also known as fibrinogen), factor II (also known as prothrombin), factor III, calcium, factor V (also known as proaccelerin), factor VI, factor VII, factor VIII, factor IX (also known as Christmas factor), factor X (also known as Stuart-Prower factor), factor XI, factor XII (also known as Hageman factor), factor XIII (also known as fibrin-stabilizing factor), von Willebrand factor, prekallikrein, high molecular weight kininogen, fibronectin, or plasminogen amongst others.

In particular embodiments, the disclosed compositions (such as tissue sealants, for example hemostatic agents or adhesives used in surgical procedures) include a drug to enhance or retard wound healing. For example, an agent that retards wound healing can be used to retard wound healing in an application where scarring of an artificially created tract, shunt, and/or fistula would not be desirable. In some embodiments, the disclosed tissue sealants include a cytotoxic or cell growth inhibitory material, for example 5-fluorouracil or mitomycin. Such cytotoxic drugs can be used in various clinical applications, such as is used in glaucoma filtration surgery, or controlling the growth of skin keloids.

In some embodiments, the disclosed compositions include an agent that promotes wound healing, for example an agent involved in tissue growth or repair, such as a growth factor. Examples of growth factors that can be used as agents in combination with the disclosed tissue sealants include, but are not limited to, transforming growth factor beta (isoforms 1, 2, or 3), basic fibroblast growth factor, epidermal growth factor, vascular endothelial growth factor, nerve growth factor, acidic fibroblast growth factor, insulin like growth factor, heparin binding growth factors, brain-derived neurotrophic factor and glial cell line-derived neurotrophic factor, platelet-derived growth factor, leukemia inhibitory factor, and combinations thereof.

In some embodiments, the compositions disclosed herein contain corticosteroids or non-steroidal anti-inflammatory agents, for example to control of post-operative inflammation, such as in uveitic eyes after intra-ocular surgery or surgery of the nervous system to control post-operative edema and inflammation.

### Preparation of a Tissue Sealant

Disclosed herein is a method for forming a composition such as a tissue sealant, for example on a tissue, such as the tissue of a subject. The disclosed methods for preparing the composition include providing whole blood, such as concentrated and/or autologous whole blood, and mixing the whole blood with an amount of an exogenous protein cross-linker effective to cross-link the whole blood into an adherent mass, such as a hemostatic mass. Typically, the whole blood and the exogenous protein cross-linker are mixed and applied to the tissue of a subject where it forms the adherent mass by forming a number of cross-links between the proteins present in the whole blood and the exogenous protein cross-linker.

In some embodiments, the whole blood used for the preparation of the disclosed composition is autologous whole blood obtained from the same subject on whom the agent will be used. By way of example, blood can be obtained from a subject, optionally concentrated, for example during the course of a surgical procedure, mixed with an exogenous protein cross linker and then applied to the subject, thereby forming the mass.

In some embodiments, the method includes mixing whole blood, such as concentrated and/or autologous whole blood, with exogenous protein cross-linker in a volume ratio from about 1 or less to about 10 or more, such as a ratio of about 10, a ratio of about 1.1, a ratio of about 1.2, a ratio of about 1.3, a ratio of about 1.4, a ratio of about 1.5, a ratio of about 1.6, a ratio of about 1.7, a ratio of about 1.8, a ratio of about 1.9, a ratio of about 2.0, a ratio of about 2.1, a ratio of about 2.2, a ratio of about 2.3, a ratio of about 2.4, a ratio of about 2.5, a ratio of about 2.6, a ratio of about 2.7, a ratio of about 2.8, a ratio of about 2.9, a ratio of about 3.0, a ratio of about 3.1, a ratio of about 3.2, a ratio of about 3.3, a ratio of about 3.4, a ratio of about 3.5, a ratio of about 3.6, a ratio of about 3.7, a ratio of about 3.8, a ratio of about 3.9, a ratio of about 4.0, a ratio of about 4.1, a ratio of about 4.2, a ratio of about 4.3, a ratio of about 4.4, a ratio of about 4.5, a ratio of about 4.6, a ratio of about 4.7, a ratio of about 4.8, a ratio of about 4.9, a ratio of about 5.0, a ratio of about 5.1, a ratio of about 5.2, a ratio of about 5.3, a ratio of about 5.4, a ratio of about 5.5, a ratio of about 5.6, a ratio of about 5.7, a ratio of about 5.8, a ratio of about 5.9, a ratio of about 6.0, a ratio of about 6.1, a ratio of about 6.2, a ratio of about 6.3, a ratio of about 6.4, a ratio of about 6.5, a ratio of about 6.6, a ratio of about 6.7, a ratio of about 6.8, a ratio of about 6.9, a ratio of about 7.0, a ratio of about 7.1, a ratio of about 7.2, a ratio of about 7.3, a ratio of about 7.4, a ratio of about 7.5, a ratio of about 7.6, a ratio of about 7.7, a ratio of about 7.8, a ratio of about 7.9, a ratio of about 8.0, a ratio of about 8.1, a ratio of about 8.2, a ratio of about 8.3, a ratio of about 8.4, a ratio of about 8.5, a ratio of about 8.6, a ratio of about 8.7, a ratio of about 8.8, a ratio of about 8.9, a ratio of about 9.0, a ratio of about 9.1, a ratio of about 9.2, a ratio of about 9.3, a ratio of about 9.4, a ratio of about 9.5, a ratio of about 9.6, a ratio of about 9.7, a ratio of about 9.8, a ratio of about 9.9, or a ratio of about 10.0 to 1.0

The disclosed methods use exogenous protein cross-linkers to effectively cross-link the protein components present in concentrated whole blood, such as concentrated autologous whole blood obtained from a subject. As discussed above, a variety of exogenous protein cross-linkers can be mixed with whole blood to effectively cross-link the protein components in the whole blood. In some embodiments, the whole blood is mixed with at least one exogenous protein cross-linker that is amine reactive, sulfhydryl reactive and/or carboxylic acid reactive, for example whole blood can be mixed with an effective amount of an exogenous protein cross-linker that contains one or more of an aldehyde, an aryl azide, a carbodiimide, a phosphine, an imidoester, an NHS-ester, a PFP-ester, a vinyl sulfone, a maleimide, or a pyridyl disulfide amongst others.

In some embodiments, the whole blood is mixed with an amount of an aldehyde, such as a di- or polyaldehyde, effective to cross-link the whole blood and form a hemostatic mass. In some embodiments, the whole blood is mixed with an amount of glutaraldehyde effective to cross-link the whole blood and form a hemostatic mass. In particular embodiments, the whole blood is mixed with a solution, such as a solution of glutaraldehyde in water, of about 1% or less glutaraldehyde to about 10% or more glutaraldehyde, such as a solution of about 1%, about 2%, about 3%, about 4%, about 6%, about 7%, about 8%, about 9%, or even about 10% glutaraldehyde.

In some embodiments, the whole blood that is mixed with an effective amount of an exogenous protein cross-linker is concentrated whole blood, such as concentrated autologous blood, for example autologous blood that is concentrated during the course of a surgical procedure. In some embodiments, the whole blood is concentrated by reducing the volume from about 1.1 times to about 10 times the original volume, such as by reducing the volume by about 1.1 times, about 1. 5 times, about 2.0 times, about 2.5 times, about 3.0 times, about 3.5 times, about 4.0 times, about 4.5 times, about 5.0 times, about 5.5 times, about 6.0 times, about 6.5 times, about 7.0 times, about 7.5 times, about 8.0 times, about 8.5 times, about 9.0 times, about 9.5 times, or even about 10 times, relative to the volume of the blood obtained from the subject.

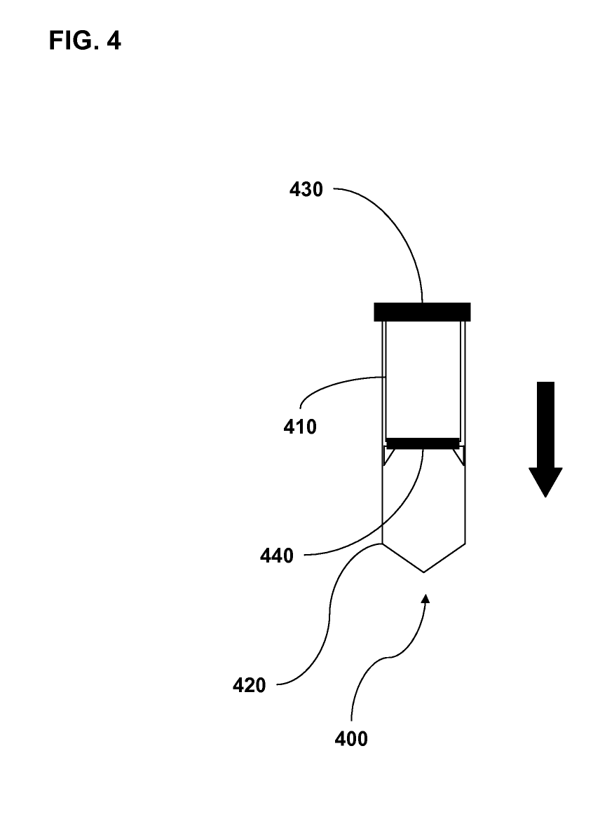

Concentrated blood can be provided by any method in the art, for example by the use of a centrifuge, filtration and/or dehydration. In some embodiments, a centrifuge is used to concentrate the blood, for example a centrifuge can be used during a surgical procedure to concentrate a subject's blood before it is mixed with an exogenous protein cross-linker and applied to the tissue of the subject. In some embodiments, a concentrator is used to concentrate the blood. An exemplary whole blood concentration device for concentrating whole blood is shown in FIG. 4. With reference to FIG. 4, in some embodiments, a blood concentrator 400, includes an inner chamber 410, an outer chamber 420, a sealing cap 430 and a membrane 440. Whole blood is placed in inner chamber 410, and when centrifugal force is applied as shown by the arrow, the water fraction of the whole blood passes through membrane 440 into outer chamber 420, effectively concentrating the whole blood in inner chamber 410. To concentrate proteins present in the whole blood, membrane 440 typically has a molecular weight cut off of about 10,000 kilodaltons to about 50,000 kilodaltons, although greater or lesser molecular weight cut offs can be used. Inner chamber 410 and outer chamber 420 are sealable and separable, for example to facilitate use of the concentrator.

In some embodiments, the concentration of total protein in the whole blood used to form the composition is from about 25 grams/deciliter to about 100 grams/deciliter, such as about 25 grams/deciliter, about 30 grams/deciliter, about 35 grams/deciliter, about 40 grams/deciliter, about 45 grams/deciliter, 50 grams/deciliter, about 55 grams/deciliter, about 60 grams/deciliter, about 65 grams/deciliter, about 70 grams/deciliter, about 75 grams/deciliter, about 80 grams/deciliter, about 85 grams/deciliter, about 90 grams/deciliter, about 95 grams/deciliter, or about 100 grams/deciliter, for example about 25 grams/deciliter to about 100 grams/deciliter, about 25 grams/deciliter to about 75 grams/deciliter, about 25 grams/deciliter to about 50 grams/deciliter, about 50 grams/deciliter to about 100 grams/deciliter, or about 75 grams/deciliter to about 100 grams/deciliter.

In some embodiments, the method of making the composition, such as a tissue sealant, includes lysing at least some of the cells present in the whole blood, such as concentrated and/or autologous whole blood. In some embodiments, the cells are lysed prior to cross-linking the whole blood, for example, prior to mixing the whole blood with the exogenous protein cross-linker. In other examples, the cells are lysed after mixing the exogenous protein cross-linker and the whole blood. The cells can be lysed be any method that disrupts the cell membrane of a blood cell, for example a mechanical means, such as a French Press or a sonicator, or chemical means, such as exposure of the cells to a effective amount of a hypotonic solution to lyse the cells. In particular embodiments, the cells are lysed by exposure to a hypotonic solution of acetic acid, such as a solution containing more than 0.1% acetic acid, for example from about 0.1% acetic acid to about 4% acetic acid, such as about 0. 1% acetic acid, about 0.2% acetic acid, about 0.3% acetic acid, about 0.4% acetic acid, about 0.5% acetic acid, about 0.6% acetic acid, about 0.7% acetic acid, about 0.8% acetic acid, about 0.9% acetic acid, about 1.0% acetic acid, about 1.1% acetic acid, about 1.2% acetic acid, about 1.3% acetic acid, about 1.4% acetic acid, about 1.5% acetic acid, about 1.6% acetic acid, about 1.7% acetic acid, about 1.8% acetic acid, about 1.9% acetic acid, about 2.0% acetic acid, about 2.1% acetic acid, about 2.2% acetic acid, about 2.3% acetic acid, about 2.4% acetic acid, about 2.5% acetic acid, about 2.6% acetic acid, about 2.7% acetic acid, about 2.8% acetic acid, about 2.9% acetic acid, about 3.0% acetic acid, about 3.1% acetic acid, about 3.2% acetic acid, about 3.3% acetic acid, about 3.4% acetic acid, about 3.5% acetic acid, about 3.6% acetic acid, about 3.7% acetic acid, about 3.8% acetic acid, about 3.9% acetic acid, or about 4.0% acetic acid, for example about 0.1% acetic acid to about 1.0% acetic acid, about 1.0% acetic acid to about 2.0% acetic acid, about 2.0% acetic acid to about 3.0% acetic acid, or about 3.0% acetic acid to about 4.0% acetic acid.

In some embodiments, other agents, such as pH modifiers, surfactants, antioxidants, osmotic agents, preservatives, drugs, and other active agents, such as exogenous active agents, for example exogenous clotting factors, are used to prepare the composition. The other agents can be added to the whole blood prior to mixing with the exogenous protein cross-linker, the exogenous protein cross-linker prior to mixing with the whole blood, or even to the composition after the whole blood and exogenous protein cross-linker are mixed.

The disclosed tissue sealant can be used on any tissue of a subject, for example the skin, bone, neuron, axon, cartilage, cornea, muscle, facia, brain, prostate, breast, endometrium, lung, pancreas, small intestine, liver, testes, ovaries, cervix, colon, skin, stomach, esophagus, spleen, lymph node, dura, bone marrow, kidney, embryonic or ascite tissue, cartilage, blood vessels, such as an artery, a vein, or even a capillary, and the like of a subject.

Tissue, such as the tissue of a subject, typically has exposed reactive groups, such as amines, sulfhydryls, and/or carboxylic acid groups, capable of cross-linking with the exogenous protein cross-linker present in the tissue sealant. Accordingly, in some embodiments, the composition cross-links with tissue, such as the tissue of a subject, on which it is placed and/or formed. By cross-linking the reactive groups present on the surface of a tissue, the disclosed composition can be used to bond portions of tissue, for example to close a wound, such as a surgical incision, and/or join two or more portions of tissue.

In some embodiments, the method of sealing tissue includes coagulating the cross-linked whole blood with a heat source to promote formation of an adherent (such as a hemostatic) mass. In some examples, the heat source is electromagnetic radiation. In some embodiments, coagulation of the cross-linked whole blood is accomplished by irradiating the cross-linked whole blood with electromagnetic radiation in a manner effective to promote the formation of a hemostatic mass, for example a hemostatic mass sealing a leak and/or joining portions of tissue. The formation of tissue sealant using electromagnetic radiation is typically achieved by the photo-oxidative effects of oxygen generating proteinaceous cross-links between the amino acid components of the tissue sealant and/or tissue.

Typically, the electromagnetic radiation that achieves protein cross-linking will generally have a wavelength from about 10 nm to about 810 nm and will be within the visual, infra red or ultra violet spectra. Exemplary methods for applying the electromagnetic radiation include coherent light, such as a monochromatic laser beam, or other form of electromagnetic radiation source. Exemplary lasers for use in the disclosed methods include blue diode lasers with an emission wavelength of about 460 nm, argon lasers with an emission wavelength of about 488 nm and about 514 nm, green diode lasers with an emission of about 532 nm, red diode lasers with an emission wavelength of about 630 nm, red diode lasers with an emission wavelength of about 660 nm, red diode lasers with an emission wavelength of about 690 nm, or tunable dye lasers such as tunable red diode lasers with an emission wavelength of about 600 nm to about 700 nm.

In some embodiments, the heat source comprises an ionized argon beam, such as from an argon beam coagulator. An argon beam coagulator is a non-contact device that conducts radio-frequency current to tissue along a jet of inert, non-flammable argon gas. A grounding pad placed under the patient allows current to flow from the tip of the probe to the tissue. Argon gas has a lower ionization potential than air and consequently directs the flow of current. The argon gas may also blow away liquid blood and other liquids on the tissue surface, enhancing visualization of the site as well as eliminating electric current dissipation in the whole blood.

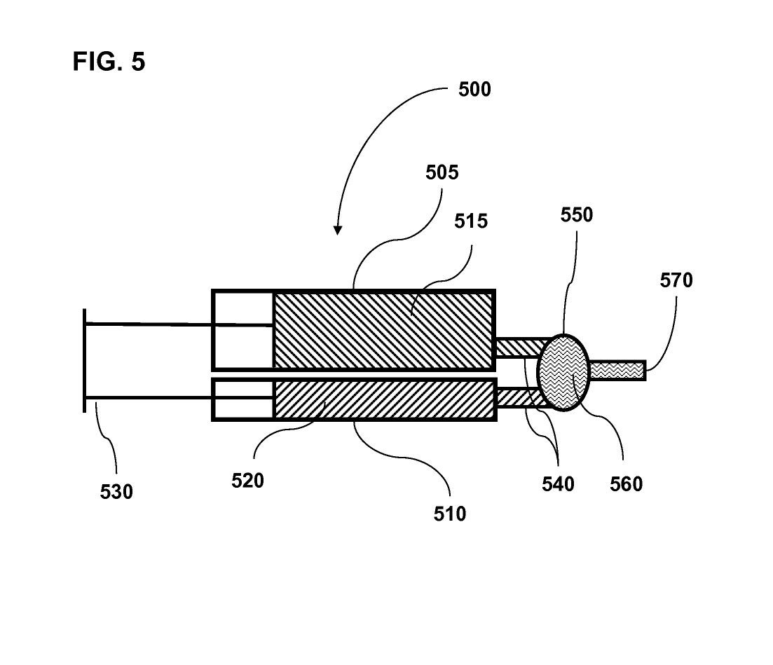

The disclosed tissue sealants can be applied to tissue of a subject by any method. For example, tissue sealants can be applied to a subject's tissue using a syringe, catheter, cannula, manually applying the composition, spraying or the like. In some embodiments, the tissue sealant is applied manually, for example as a paste that is smeared or brushed on a surface, such as the surface of a tissue, such as the tissue of a subject. In some embodiments, a proportionally sized double-barreled syringe equipped with a mixing tip can be used to deliver cross-linkable whole blood in a volume ratio relative to the exogenous protein cross-linker of between about 10:1 and about 1:1. A particularly suitable device is depicted in FIG. 5. With reference to FIG. 5, a device 500 is constructed from two syringe bodies 505, 510, that can be filled with whole blood 515 and exogenous protein cross-linking solution 520. A plunger 530 has dual plunger heads, one of which is disposed in each of syringe bodies 505, 510 for controlled reciprocation therein. When plunger 530 is depressed, the whole blood 515 and protein cross-linking solution 520 is transferred via tubes 540 into mixing chamber 550 where it mixes with a protein cross-linker 560. The mixed whole blood and exogenous protein cross-linker 560 can then be applied to the tissue of a subject via applicator tip 570. By altering the volumes of syringe bodies 505, 510 the ratio of whole blood to exogenous cross-linker can be easily varied. In another embodiment, a applicator tip can be replaced by a spray nozzle tip, such as that sold under the trade name TISSEEL® (Immuno AG, Vienna, Austria). With a spray nozzle fitted to the double-barreled syringe, an atomized spray of tissue sealant is released upon syringe plunger depression.

### Use of Tissue Sealant

The disclosed compositions are useful for a wide variety of medically related purposes, for example in medical (including surgical) procedures where the practitioner desires to seal, close, appose or join portions of soft tissue, for a example portions of skin, neuron, axon, cartilage, cornea, muscle, fascia, brain, prostate, breast, endometrium, lung, pancreas, small intestine, liver, testes, ovaries, cervix, colon, skin, stomach, esophagus, spleen, lymph node, dura mater, bone marrow, kidney, embryonic or fetal tissue, cartilage, blood vessels, such as an artery, a vein or even a capillary, and the like of a subject. The disclosed tissue sealants can reduce operative time and prevent complications associated with surgical intervention (such as hemorrhage, fluid leaks, air leaks, and/or fistula formation) which may result in reduced postoperative discomfort and hospital stay for patients.

The disclosed tissue sealants have particular application as hemostatic agents, for example as agents to inhibit hemorrhage from the tissue of a subject, such as from a blood vessel organ of a subject. The tissue sealants disclosed herein can be used to seal or fill defects, for example voids or holes, in tissue, and therefore find use as hemostatic agents. The disclosed tissue sealants may serve to stop or staunch the flow of fluid, for example blood, through ruptured vessels, such as arteries, veins, capillaries and the like. For example, the disclosed sealants can be applied at the site of a defect, such as a defect in a vessel and/organ, whereby it will set via protein cross-linking of the whole blood and seal the defect, for example by forming an artificial clot in the subject at the site of application sufficient to block flow from blood vessels or damaged surfaces, such as wounds. It is appreciated that the sealant can be applied from within a vessel or externally, optionally with pressure applied to the vessel hole through an inflatable catheter or external pressure, for example when used to staunch bleeding, such as bleeding from blood vessels, such as an artery, a vein, or even a capillary. The sealant can be applied in layers to gradually fill in a defect or gradually strengthen the wound.

In some embodiments, the tissue sealants are used to bond tissue, for example to close a wound, such as occurs during an injury or even as part of a surgical procedure, such as in closing a surgical incision. In some embodiments, the disclosed tissue sealants are used to seal a fluid or gas leak in a tissue of a subject, for example a leak in a blood vessel or lung of a subject.

The disclosed tissue sealants can be used in medical applications where precise adhesion is necessary and where the application of sutures or staples is inconvenient or less effective than a tissue sealant. Alternatively, the disclosed tissue sealants can be used in conjunction with other securements, such as sutures, staples and/or clips, for example to provide a better cosmetic closure, such as to provide a smooth surface on top of the suture surface, or to provide protection against fluid or gas leaks, for example in the joining the ends of two portions of blood vessels, for example as reinforcement for a sutured anastomosis, thus rendering it water tight and bacteria tight.

The tissue sealants are particularly suitable for use in surgery, for example, in surgical operations or maneuvers concerning the eye, small vascular tissue, gastro-intestinal tract, nerve sheaths, small ducts for example, urethra, ureter, bile ducts, thoracic duct) or even the inner ear, teeth or gums, anastomosis and coronary artery bypass graft surgery, sealing livers following split liver resection transplants, severe bleeding in liver, spleen, lung, heart, bone, and brain tissues; sealing grafts, ruptured aorta, ruptured vena cava, torn right ventricle as a result of re-operation, dissected aorta, artificial heart valves of biological, autologous or mechanical construction, left ventricular assist devices, long-term catheters, infusion ports, and percutaneous access device ports or otherwise as an adjunct or substitute for surgical sutures or staples.

The tissue sealants disclosed herein can be used to seal openings, such as wounds, for example incisions formed in any vascular organs such as the kidneys, the liver, the spleen, the heart, the brain, and the stomach. For example raw cut surfaces of soft tissues such as liver and kidneys cannot be isolated and readily secured by conventional techniques such as suturing. A surgeon can use the disclosed tissue sealant to seal spleen and liver lacerations, which is important in the preventing intraperitoneal complications, such as infection, abscess formation, and sepsis which may lead hemorrhage, bile leakage, and fluid accumulation.

In certain embodiments, the disclosed tissue sealants are used to close a gas leak, such as a gas leak in a lung of a subject. For example, typically during pulmonary resection and decortications hemorrhage and air leaks can result, which can form bronchopleural fistulae that may result in high rates of mortality. Such complications can be minimized with the use of the disclosed sealant to close blood and air leaks during the procedure. Other applications of the disclosed tissue sealants include lung transplantation and bronchial anastomosis, in which the tissue sealant prevents air leakage from the anastomosis. The sealant is also useful in reestablishing the integrity of the thoracic cavity and helping re-inflate a collapsed or partially collapsed lung.

The disclosed tissue sealants can be used to close large wounds and tissue defects, for example as in filling in a defect caused by debridement. In another example, the disclosed sealants are used as an artificial skin or covering agent to cover large, oozing surfaces inside or outside the body, for example to attach skin grafts. In some examples, the sealant is used to inhibit bleeding after debridement and as adjuncts in surgery that require flaps. Skin grafting is the simplest and most effective method used to resurface large burn wounds. The graft initially adheres to its new bed by a thin layer of fibrin and nourishment of the graft occurs by plasmatic imbibition. Further ingrowth of blood vessels and fibrous tissue from the wound results in permanent adherence of the graft to its recipient site known as graft “take.” This process can be hindered by collection of blood between the graft and bed, by shearing and by infection. The face is highly vascular and diffuse bleeding is difficult to control following burn wound excision. Traditionally, to overcome the problem of hematoma, the grafts are meshed to enable any fluid collection to drain. Unfortunately meshing produces scarring which impairs the final cosmetic result. Careful suturing can minimize shearing, but takes time, may promote bleeding and also leaves scars. The tissue sealants disclosed herein can be used to improve adhesion of a graft to an underlying tissue bed, and thereby improve graft survival while minimizing complications.

### EXAMPLES

### Example 1

### Functional Characterization of the Hemostasis Using Concentrated Autologous Blood

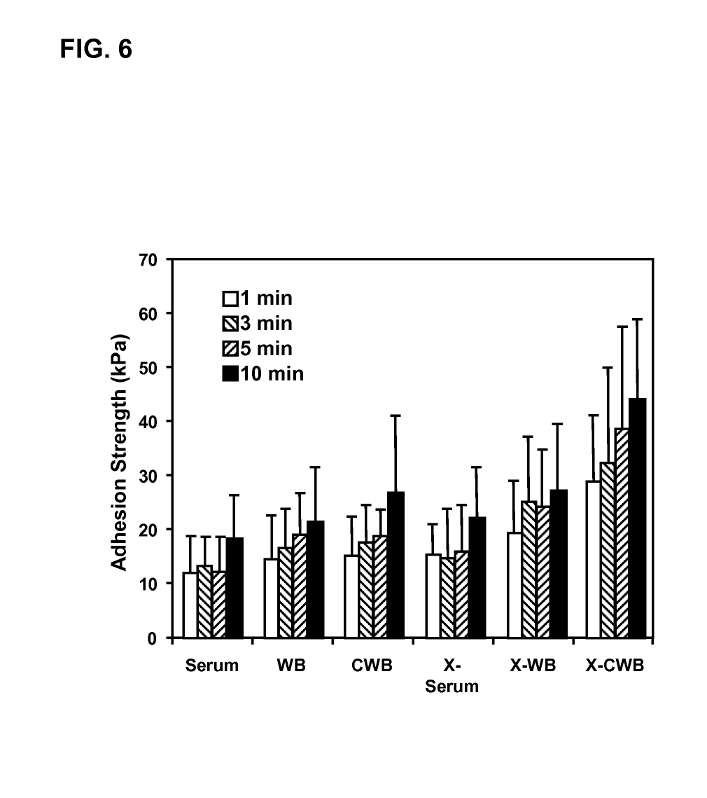

This example describes the characterization of autologous blood as a tissue sealant used as a hemostatic agent. Fresh whole blood was collected into 300 milliliter blood bags with sodium citrate from porcine from other studies prior to sacrifice. A blood sample drawn from the bags was sent to the laboratory for chemical analysis and blood count. The blood samples were then divided into 50 milliliter sterile centrifugation tubes and centrifuged for about 15 to about 20 minutes at 3000 rpm. During centrifugation, the blood separated into two layers, which included a top supernatant layer of serum and bottom layer of cellular components, which was mainly red blood cells (RBCs). After the supernatant serum was removed, the cellular components were re-mixed and the samples were analyzed to determine the concentration of protein in the blood cellular fraction. The concentration of protein in the concentrated blood samples averaged 39±12 grams/deciliter (ranging from about 30 grams/deciliter to about 50 grams/deciliter, hemoglobin plus total serum proteins). The total protein concentration from fresh unconcentrated porcine blood averaged 13±5 grams/deciliter (ranging from about 13 grams/deciliter to about 18 grams/deciliter, hemoglobin plus total serum proteins). The concentrated blood was mixed and cross-linked with a solution of 8% glutaraldehyde at a four to one ratio to form a pasty-to-solid platform within a few minutes in in vitro tests. In vitro tests showed that the results of adhesion strength of the cross-linked blood and serum were varied (see FIG. 6).

The concentrated whole blood also was used as solder in association with argon beam coagulation in liver resection for hemostasis in a porcine model. In vitro screening of effectiveness of early formula combinations involved testing of tensile and sheer strength using uni-axial test methods. The general adhesiveness of the concentrated blood with/without cross-linking reagents was tested with ASTM F2258-03 Standard Test Method for Strength Properties of Tissue Adhesives in Tension. Thirty milliliters of fresh porcine blood was collected from an arterial line using a 60 milliliter heparin coated syringe. The blood was immediately transferred into a 50 milliliter sterile centrifuge tube (Fisher Scientific, and centrifuged at 3000 rpm for 15 minutes. The blood cells and serum were separated with the centrifugation force at ratio of 1 to 2 by volume. The supernatant serum was transferred into other tube. After the serum was removed, the remaining blood cells were well mixed by shaking. The blood was divided and 1%, 2%, or 4% acetic acid was added to individual concentrated blood samples to induce cytolysis. Samples of fresh porcine blood, serum, and the concentrated blood were divided into two groups, the first group had 8% glutaraldehyde added to the samples in a 1 to 4 ratio by volume; the second group did not receive glutaraldehyde treatment.

### Example 2

### Tissue Adhesive Testing

This example describes the procedures used to test the adhesive strength of various cross-linked and non cross-linked blood products. The adhesion test was conducted by applying a 50 microliter blood or serum sample between a cover plate and a loading plate. A 200 millimeter PVC surface area was brought into contact with the concentrated blood and loaded at a rate of 10 Newton/second to 10 Newtons (50 Kpa) and held for 1, 3, 5, or 10 minutes. The adhesion-test surface was then pulled away from contact with the concentrated blood at a rate of 1 millimeter/second and the adhesion strength (kPa) is determined. Student's t-Test and ANOVA analysis were used to determine statistical significant differences between the various groups of different concentrations of blood, cross-linking reagent, and time points (see FIG. 6). With reference to FIG. 6, the adhesive strength of serum, whole blood (WB), concentrated whole blood (CWB), cross-linked serum (X-serum), cross-linked whole blood (X-WB), cross-linked concentrated whole blood (X-CWB) was determined at time points 1 minute, 3, minutes, 5 minutes, and 10 minutes. As shown in FIG. 6, the cross-linked whole blood and cross-linked concentrated whole blood demonstrated unexpected superior adhesive strength relative to the uncross lined species and cross-lined serum.

The change of cellular morphology was also evaluated after cytolysis and cross-linking processes. The acceptance criteria of the in vitro adhesion testing was an adhesion strength greater or equal to 30 kPa, which is the same criteria for the FDA approved Fibrin Adhesives.

### Example 3

### Hemostasis Following Liver and Kidney Resection Using Concentrated Autologous Blood

This example describes the testing of cross-liked concentrated blood as a tissue sealant during a surgical resection. Concentrated blood mixed with 8% glutaraldehyde at 4 to 1 ratio was tested for the ability to induce hemostasis in an acute hepatic injury and laparoscopic partial nephrectomy model in swine with a two-chamber delivery device. Concentrated blood (48.0 grams/deciliter, hemoglobin plus total serum protein) cross-linked with 8% glutaraldehyde was tested to control bleeding from parenchymal organs.

A juvenile crossbred domestic swine (body weight 52 kilograms) was studied in accordance with the guidelines of the 1996 National Research Council Guide for the Care and Use of Laboratory Animals and applicable federal regulations. In brief, after the induction of general anesthesia the swine was prepped with sterile technique and placed in the dorsal recumbent position in order to introduce a 14 gauge Veress needle at midline above the umbilicus following abdomen insufflation. Sixty milliliters of blood was withdrawn prior to the operation using a 60 milliliter syringe containing 5 milliliters of heparin. The blood was centrifuged in centrifuge tubes at 3000 rpm for 15 minutes. The supernatant serum was removed from the tubes and the cellular components were aspirated into 5 milliliter syringes for surgical use. After pneumoperitoneum was established with a maximum of 15 millimeters of Hg CO₂ pressure, the swine was placed in a left flank position and underwent a laparoscopic left lower-pole partial nephrectomy via trans peritoneal approach. Ten minutes prior to the operation procedure, the animal was given 5000 units of intravenous heparin and a 500 milliliter fluid bolus of Ringer's lactate solution. The Activated Clotting Time (ACT) was checked, and if greater than 200 seconds, the operation proceeded. If the ACT was less than 200 seconds, additional heparin was given. 1000 units of heparin were given intravenously every 30 minutes to maintain the ACT greater than 200 seconds during the operation.

The operation was performed using a four-portals fashion detailed as follows: the first 5 millimeter port at the midclavicular line 3 inches above umbilcus; the second 5 millimeter at one inch below umbilicus in the line joining to the anterior superior iliac spine; A 10 millimeter port at 2 inches horizontal above umbilicus for a 45° degree laparoscope. The third 5 millimeter port at midway between the second 5 millimeter port and the 10 millimeter port was placed for delivery of the sealant. The lower-pole of the kidney was mobilized after incision and reflection of the posterior peritoneum. The renal artery and vein were isolated and the ureter was identified. A laparoscopic Satinsky clamp was used to occlude the renal artery and veins and then the lower-pole of the kidney was amputated using cold scissors. In every case transection of the collecting system was confirmed by direct vision. After transection of the lower-pole the animal underwent treatment of using concentrated autologous blood with 8% glutaraldehyde. The entire transected renal parenchymal surface was treated with the cross-linked concentrated autologous blood while the renal vascular occlusion was applied using a laparoscopic needle applicator passed through a 5 millimeter port. Following removal of the clamps, the transected surface was inspected. Initial hemostasis was achieved in two applications in about 5 minutes. The abdomen was then deflated and an additional 30 minutes was given to observe the stability of the repairs. After 30 minutes the renal parenchyma sunace was re-inspected and hemostasis was verified. The pig was then turned to the supine position. A midline epigastric approach was used to expose the liver. The median left lobe of the liver was transected using the finger fracture surgical technique so that the vessels of the resection area larger than 5 millimeter in diameter can be ligated along the demarcation line. The cross-linked concentrated blood was applied via the prototype device to the resection surface without vascular occlusion. Complete hemostasis was achieved after 3 minutes with 3 repeated applications of concentrated autologous blood mixed with 8% glutaraldehyde.

The study demonstrated the feasibility of cross-linked autologous blood to control significant bleeding from the liver and renal resection in laparoscopic and open surgical procedures.

### Example 4

### Hemostasis Following Liver and Kidney Resection Using Concentrated Autologous Blood and Argon Beam Coagulation

This example describes the use of concentrated autologous blood as a surgical solder. Autologous blood (60 milliliters) was acquired prior to operation from the same pig using a 60 millimeter syringe containing 5 millimeter of heparin. The blood was centrifuged at 3000 rpm for 15 minutes in centrifuge tubes. The supernatant serum was removed from the tubes and the cellular components as a concentrated blood were aspirated into 10 millimeter syringes for surgical use. The surgical procedures were described as in the previous example. In the laparoscopic procedure, after transection of the lower-pole of the kidney, the transected raw surface was coated with a thin layer of the concentrated blood by using a laparoscopic needle applicator passed through a 5 millimeter port. The concentrated blood layer was then “soldered” to the renal parenchyma surface using argon beam coagulation (FORCE ARGON™ II, Valleylab, Boulder, Colo.) with a laparoscopic applicator (OPTI4™ laparoscopic handset with nonretractable hollow electrode E2786-28, Valleylab, Boulder, Colo.) in the “fulgurate” setting at 75 W and an argon flow rate of 4 liters/minute. The argon beam was applied to the transected parenchyma surface of the kidney while the renal vascular occlusion was applied. Complete hemostasis was achieved with 2 repeated applications and lasted for more than 1 hour at de-pneumoperitoneum. In open hepatectomy, hemostasis was obtained by 2 re-applications in the left median lobe of this animal using the same argon beam coagulation setting.

### Example 5

### Test for Cross-Linking and/or Coagulating Techniques in Concentrated Autologous Blood

This example describes testing of combinations of the concentrated blood and cross-linking and/or coagulating reagents. The combinations are evaluated via uni-axial in vitro tests. The efficacy of the combinations is tested to mimic in vitro conditions. Examples of cross-linking and coagulating reagents tested include glutaraldehyde, resorcinol, thrombin, and fibrinogen. The concentration of blood is between about 30.0 grams/deciliter and about 60.0 grams/deciliter, such as about 38.0 grams/deciliter, about 45.0 grams/deciliter, or about 52.0 grams/deciliter. In the in vitro test, in a standardized experimental setting, pig skin or PVC film is used for evaluation of both tensile and shear strength of the combination formula of concentrated blood and selected cross-linkers. Tensile strength is defined as the maximum elongating force in the plane of the bond, which the sealant material can withstand without tearing. Shear strength is the maximum force that can be applied parallel to the plane of the sealant material. Tensile strength measurements are made by using fresh pig skin cut into strips. The test area is about 1 square centimeter. The mounted grafts, as well as the cross-linking reagent and the concentrated blood mixture, are warmed in a 37° C. Ringer's solution bath. The skin grafts are joined together by separately applying 0.125 milliliters of each mixture component to the test areas of opposite strips and then applying uniform and light pressure for 5 seconds. The mounted and joined skin grafts are incubated at time periods ranging from about 5 minute to about 90 minutes in a Ringer's solution bath before testing. PVC samples are prepared in a similar manner. In shear strength measurements, PVC film or pig skin are cut into 2.5 centimeter×0.5 centimeter strips. Each mixture component is separately applied to the one side of the strip measuring 1 square centimeter. The combination of concentrated blood and cross-linking reagents are mixed by joining the strips together gently to form a clot. For both measurements, the strips are separated by moving the crosshead of the load-cell (carrying one strip) away from the other load plane loaded with other strip. The material strength tester (VITRODYNE V1000 UNIVERSAL TESTER™, Chatillon, Greensboro, N.C.) is operated at a constant speed of 5 centimeters/minute using a 2 kilogram load cell. The maximum breaking strength (tensile strength) of the mixture is measured in grams per 6.25 square centimeter. The results are expressed in Pascal. The force is recorded as a load-extension curve and the highest force achieved is used as the maximum breaking strength. All samples for each tested group are prepared on the day of the test. In the first series of tests, the tensile and shear strength of the tissue sealant using fresh pig skin and PVC film as templates are compared. To determine the minimum time required to establish the maximum attainable adhesion strength of mixtures, in the second series of test, shear strength of the mixtures as a function of different protein concentration, ratio of the mixture and the incubation time is determined. To further characterize the effect of concentration of blood protein on the sealant properties of the mixtures, shear strength measurements are determined as a function of protein concentration. Blood protein are prepared using different concentrations (from about 35.0 to about 55.0 grams/deciliter) to mix with 10% glutaraldehyde, 45 milligrams/milliliter thrombin and 35 milligrams/milliliter fibrinogen, and 2% resorcinol, respectively.

### Example 6

### Optimization of the Hemostatic Technique of Using the Concentrated Autologous Blood for Hemorrhage Control of Parenchymal Organs

This example describes efficacy tests for the selected combinations tested in the previous example. Fresh liver samples obtained from a local supermarket are filleted into large area sheets less than 5 millimeter thick with the parenchymal surface left intact. The liver sheet is cut into dog-bone shaped pieces approximately 4 centimeter long and 1 centimeter wide in the middle and 2 centimeter wide at the ends. The liver sections are kept hydrated with phospho-buffered saline (PBS) saturated paper towels and covered until tested for tensile failure. Twenty samples are tested for each selected combination. The dog-bone liver samples will be cut in the middle and joined with the mixture of combination for different time. The joined samples are glued (prism 4081, Loctite Co. CT) to aluminum plates with a 3 millimeter gap between plates centered on the joined incision and then tested under tension to failure on a materials tester (No. 300213-01, MTS MINI BIONIX® II Eden Prairie, Minn.). The samples are pulled to a displacement of 1.5 centimeter to ensure failure at a rate of 2 millimeters/second.

### Example 7

### Optimization of Blood Concentration Techniques

This example describes the concentration of blood with a filter-centrifugation system. The refractive index is measured for 10 different protein concentrations at 24° C. and the data fit to a calibration curve. The equation of the calibration curve is as follows:

% concentration=575 (nprotein−nwater),

Where nprotein is the measured refractive index of the protein solution and nwater=1.333 is the refractive index of water at 24° C. The starting protein concentrations are 80, 90, 100, 110, 120, 130, 140, 150, 160, and 170 g/L. To determine the time and centrifugation speed required to obtain the three required concentrations, 380 g/L, 450 g/L and 520 g/L two experiments are performed. The effect of centrifugation speed with respect to time at three different speeds, 3000 rpm, 4000 rpm, 5000 rpm and the effect of varying time at constant centrifugation speeds is evaluated. In this study, the fresh porcine whole blood is obtained from Lampire Biologicals Laboratory (Pipersville, Pa.).

### Example 8

### Development of a Filter-Centrifugation Device for Blood Concentration

This example describes an exemplary centrifugation tube for concentrating blood for use as a tissue sealant. In the operation room preparation, all tools and devices used in the surgical field are handled under sterile conditions. Contamination is a risk when using an open tube during the centrifugation procedure. To account for this, a device with double-chamber filter-centrifugation system was designed (FIG. 4) to obtain the adequate protein concentration from autologous blood in a closed sterile environment. The device provides a system to separate extra water from autologous blood by centrifugation. The final protein level of the concentrated autologous blood will be varied from 380 g/L to 520 g/L depending on the application. The design concept of the filter-centrifugation system device came from our previous study on albumin concentration procedures. The molecular weight of human hemoglobin is very close to albumin, which is about 68 KDa. Before hemolysis, the hemoglobin is kept in a 7 μm diameter RBC. The filter-centrifugation system includes an inner chamber with a 10,000-50,000 MWCO filtration membrane (Amicon, Millipore Inc, Mass.) and an outer chamber. Both chambers are sealed respectively and can be separated. Through the filter-centrifugation procedure, the high molecular weight components (MW<12 KDa-14 KDa) and extra water will be filtered out into the outer chamber. The inner chamber with the concentrated blood can be extracted for surgical use.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

We claim:

1. A method for forming a tissue sealant, comprising:
providing concentrated whole blood; and
mixing the concentrated whole blood with a effective amount of an exogenous protein cross-linker to cross-link the concentrated whole blood into a tissue sealing composition.

2. The method of claim 1, wherein the concentrated whole blood is mixed with the exogenous protein cross-linker in a volume of concentrated whole blood to exogenous protein cross-linker ratio from about 1 to 1 to about 10 to 1.

3. The method of claim 1, wherein the exogenous protein cross-linker comprises an aldehyde.

4. The method of claim 3, wherein the aldehyde comprises glutaraldehyde.

5. The method of claim 1, further comprising lysing cells present in the concentrated whole blood prior to cross-linking the concentrated whole blood.

6. The method of claim 5, the comprising lysing cells present in the concentrated whole blood prior to mixing the concentrated whole blood with the exogenous protein cross-linker.

7. The method of claim 5, wherein lysing cells present in the concentrated whole blood comprises adding an effective amount of a hypotonic solution to the concentrated whole blood, thereby lysing the cells.

8. The method of claim 1, wherein the concentration of total protein in the concentrated whole blood is from about 25 grams/deciliter to about 100 grams/deciliter.

9. The method of claim 8, wherein the total protein is extracellular protein (serum protein) and intracellular protein present in whole blood.

10. The method of claim 1, further comprising coagulating the cross-linked concentrated whole blood with a heat source to promote formation of the hemostatic mass.

11. The method of claim 1, further comprising mixing the concentrated whole blood with one or more exogenous coagulation factors.

12. The method of claim 1, wherein the tissue sealant is formed on the tissue of a subject and wherein providing the whole blood comprises providing autologous whole blood obtained from the subject.

13. A method for sealing tissue in a subject, comprising
providing whole blood;
mixing the whole blood with a effective amount of an exogenous protein cross-linker, wherein the effective amount of the exogenous protein cross-linker is sufficient to cross-link the whole blood, thereby producing a mixture; and
applying the mixture to the tissue of the subject to form a cross-linked hemostatic mass, thereby sealing the tissue in the subject.

14. The method of claim 13, wherein the whole blood is mixed with the exogenous protein cross-linker in a volume of whole blood to exogenous protein cross-linker ratio from about 1 to 1 to about 10 to 1.

15. The method of claim 13, wherein the whole blood is concentrated whole blood.

16. The method of claim 13, wherein the exogenous protein cross-linker comprises an aldehyde.

17. The method of claim 16, wherein the aldehyde comprises glutaraldehyde.

18. The method of claim 13, further comprising lysing cells present in the whole blood prior to cross-linking the whole blood.

19. The method of claim 18, the comprising lysing cells present in the whole blood prior to mixing the whole blood with the exogenous protein cross-linker.

20. The method of claim 18, wherein lysing cells present in the whole blood comprises adding an effective amount of a hypotonic solution to the whole blood, thereby lysing the cells.

21. The method of claim 13, further comprising coagulating the cross-linked whole blood with a heat source to promote formation of the hemostatic mass.

22. The method of claim 13, further comprising mixing the whole blood with one or more exogenous coagulation factors.

23. The method of claim 13, wherein providing the whole blood comprises providing autologous whole blood obtained from the subject.

24. The method of claim 13, wherein sealing the tissue comprises sealing a fluid or gas leak in a tissue of a subject or adhering sections of tissue to one another.

25. The method of claim 13, wherein sealing the tissue comprises sealing the tissue during a surgical procedure.

26. The method of claim 13, wherein sealing the tissue comprises forming a hemostatic seal.

27. A tissue sealant, comprising concentrated whole blood and an effective amount of an exogenous protein cross-linker to cross-link the concentrated whole blood.

28. The tissue sealant of claim 27, wherein the concentrated whole blood is present in a volume of concentrated whole blood to exogenous protein cross-linker ratio from about 1 to 1 to about 10 to 1.

29. The tissue sealant of claim 27, wherein the exogenous protein cross-linker comprises an aldehyde.

30. The tissue sealant of claim 27, wherein the concentrated whole blood comprises cytolysed concentrated whole blood.

31. The tissue sealant of claim 27, wherein the concentration of total protein in the concentrated whole blood is from about 25 grams/deciliter to about 100 grams/deciliter.

32. The tissue sealant of claim 27, further comprising one or more exogenous coagulation factors.
